(19) United States
(12) Patent Application Publication
(10) Pub. No.: US 2019/0115102 A1
(43) Pub. Date: Apr. 18, 2019

(54) ORTHOPEDIC VENDING MACHINE

(71) Applicants: Joseph Jin, Fort Lauderdale, FL (US); Ravi K. Ponnappan, Moorestown, NJ (US)

(72) Inventors: Joseph Jin, Fort Lauderdale, FL (US); Ravi K. Ponnappan, Moorestown, NJ (US)

(21) Appl. No.: 16/160,614

(22) Filed: Oct. 15, 2018

Related U.S. Application Data
(60) Provisional application No. 62/572,750, filed on Oct. 16, 2017.

(51) Int. Cl. G16H 40/40; G16H 10/60

(52) U.S. Cl. CPC G16H 40/40; G16H 10/60

(57) ABSTRACT

A medical device dispenser is disclosed. The medical device dispenser comprises a storage compartment, a dispensing mechanism and a computer. The storage compartment functions to store a plurality of medical devices. The dispensing mechanism functions to dispense at least one of the plurality of medical devices. The computer includes a processor and a memory having stored thereon computer instructions executable by the processor. The computer instructions are executable to receive patient specific data; to receive a unique device identification associated with each of the plurality of medical devices; to determine at least one of the plurality of medical devices to output based on the patient specific data; and to associate the unique device identification of the determined medical device with the patient specific data.

### CROSS-REFERENCE TO RELATED APPLICATION

This application claims priority under 35 U.S.C. § 119(e) to U.S. Provisional Application Ser. No. 62/572,750, filed Oct. 16, 2017, and entitled “Orthopedic Vending Machine,” the disclosure of which is hereby incorporated by reference in its entirety for all purposes.

### BACKGROUND OF THE INVENTION

Implants are generally used during orthopedic surgeries to cure, treat and/or mitigate human ailments are maladies. Generally, an orthopedics sales representative is on sight during a surgical procedure to provide surgeons with the necessary medical devices, such as implants and instruments, for the particular procedure.

Such medical devices are regulated by the Food and Drug Administration (“FDA”) which require all medical devices to have a Unique Device Identification (“UDI”) which is assigned by system operated by an FDA-accredited issuing agency and part of which is recorded in an FDA-administered Global Unique Identification Database (“GUDID”). The goal of such guidelines is to monitor the use and administration of medical devices in an effort to protect patients and users.

### BRIEF SUMMARY OF THE INVENTION

The following exemplary embodiments of the present invention relate generally to a medical device dispensing machine.

In accordance with an exemplary embodiment, the subject disclosure provides for a medical device dispenser comprising a storage compartment, a dispensing mechanism and a computer. The storage compartment functions to store a plurality of medical devices. The dispensing mechanism functions to dispense at least one of the plurality of medical devices. The computer includes a processor and a memory having stored thereon computer instructions executable by the processor. The computer instructions are executable to receive patient specific data; to receive a unique device identification associated with each of the plurality of medical devices; to determine at least one of the plurality of medical devices to output based on the patient specific data; and to associate the unique device identification of the determined medical device with the patient specific data.

### BRIEF DESCRIPTION OF THE SEVERAL VIEWS OF THE DRAWINGS

The foregoing summary, as well as the following detailed description of the exemplary embodiments of the disclosure, will be better understood when read in conjunction with the appended drawings. For the purpose of illustrating the subject disclosure, there are shown in the drawings exemplary embodiments of the disclosure.

It should be understood, however, that the disclosure is not limited to the precise arrangements and instrumentalities shown.

In the drawings:

FIG. 1 is a front elevational view of an orthopedic vending machine, in accordance with an exemplary embodiment of the present disclosure; and

FIG. 2 is a schematic view of the computer architecture of the orthopedic vending machine of FIG. 1.

### DETAILED DESCRIPTION OF THE INVENTION

Reference will now be made in detail to the various exemplary embodiments illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below, front, real, and diagonal, are used with respect to the accompanying drawings. The terms “right,” “left,” “front,” and “rear” are used to describe positions relative to an operator. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially,” as used herein, shall mean considerable in extent, largely, but not wholly, that which is specified, or an appropriate variation therefrom as is acceptable within the field of art.

Throughout this disclosure, various aspects of the exemplary embodiments can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments.

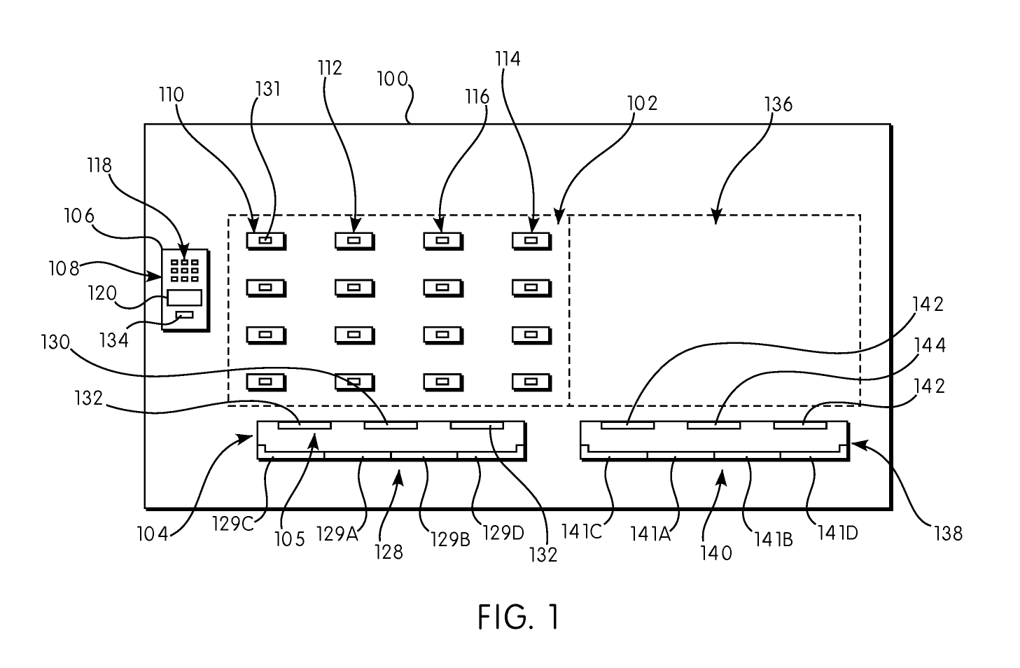

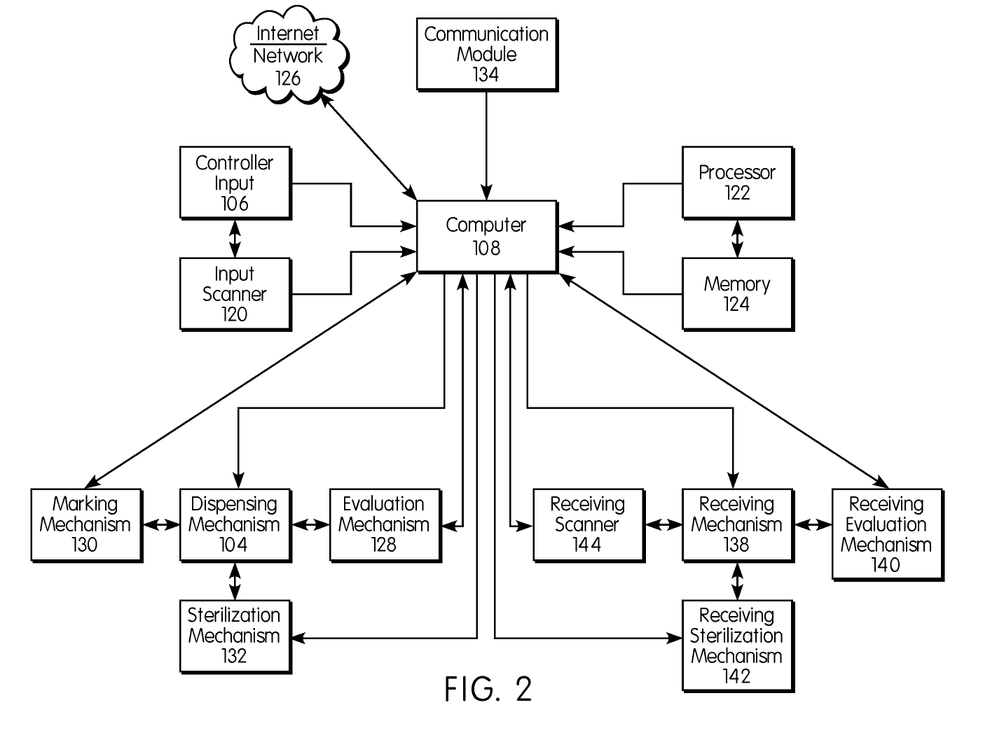

Referring now to FIGS. 1 and 2, there is illustrated a medical device dispenser 100 in accordance with an exemplary embodiment of the subject disclosure. The medical device dispenser 100 includes a storage compartment 102, a dispensing mechanism 104, a controller 106 and a computer 108.

The storage compartment 102 is an enclosed spaced for housing a plurality of medical devices such as implants 110, trial implants 112, instruments 114, and/or kits 116 that include a set of, e.g., instruments and/or implants. Other medical devices applicable to the subject invention include, e.g., devices related to the brain, spine, central nervous system or skeletal system. In an exemplary embodiment, the storage compartment 102 includes a sterile housing for housing a plurality of medical devices.

In an exemplary embodiment, the plurality of medical devices are implants and/or instruments that are required to be sterile such as the implants 110, trial implants 112, instruments 114, and kits 116.

In an exemplary embodiment, the storage compartment 102 is divided into a plurality of individual storage sections for separating the implants 110, trial implants 112, instrument 114 and kits 116 from each other. To maintain sterility, the storage compartment 102 is configured as a hermetically sealed storage compartment. Additionally, the implants 110, trial implants 112, instruments 114, and kits 116 may be packaged in sterile packaging before being stored in the storage compartment 102.

The implants 110, trial implants 112, instruments 114 and kits 116 are intended to be used during an orthopedic operation. The kit 116 may include, e.g., a series of trial implants of different sizes, another kit may include a different series of trial implants of different sizes from the previous kit, yet another kit may include a series of instruments typically used during operation, and so forth.

The dispensing mechanism 104 is a mechanism for dispensing one of the implants 110, trial implants 112, instruments 114 and kits 116 from the storage compartment 102. In an exemplary embodiment, the dispensing mechanism 104 includes a central opening 105 that is in communication with an area outside the medical device dispenser and in operative communication with a retrieval mechanism for retrieving one of the medical devices. For example, the dispensing mechanism 104 can include a robot arm that grabs one of the medical devices, wherein the medical devices are independently sealed in sterile packaging. The dispensing mechanism 104 may alternatively include a movable shaft and elevator mechanism that movably aligns with one of the individual storage sections for transporting to the central opening 105. In another exemplary embodiment, the individual storage section may each have an independent latch that can be independently opened for accessing the individual medical device therewith.

The controller 106 is a mechanism for operating the medical device dispenser 100. The controller 106 includes an input 118 for inputting patient specific data, which can include, without limitation, patient image data, into the medical device dispenser 100. The controller 106 can be, e.g., an input scanner 120, keyboard, USB or other communication means configured to receive patient specific data. The controller 106 can further be configured to receive data specific to the medical device and/or instruments necessary for a surgery and/or patient. The controller 106 further includes a display for displaying the operations of the medical device dispenser 100.

As shown in FIG. 2, the computer 108 includes a processor 122 and a memory 124. The computer 108 is configured to receive patient specific data from the controller 106 and operates the medical device dispenser 100 in response to the patient specific values. The computer 108 is further configured to store in its memory 124 medical information of the medical devices stored in the medical device dispenser 100 and dispensed for a particular surgery. Such medical information includes the sizes of the implants, the types of instruments, the unique device identification (UDI) of the medical device, and so forth.

Patient specific data can include, e.g., a patient's age, weight, height, image data and so forth. Patient image data can include, e.g., an X-ray, a CT scan, an MRI scan, and so forth. Patient specific data can further include a selection of a specific size, type, etc., of an implant, trial implant, or instrument to be used on the patient.

The computer 108 can further be in communication with a network 126, such as the internet, or a central server located in, e.g., a hospital or a medical clinic where the medical device dispenser 100 is operating, for transmitting operational metrics of the medical device dispenser 100, such as patient specific data, and/or instruments and/or implants used in the surgery, thereto. The network 126 can also be a cloud based network. The network 126 may also include patient specific data which may be accessed through the medical device dispenser 100. The network 126 may also be used to order or request maintenance for the medical device dispenser, such as refilling a stock of a specific medical device.

The computer 108 includes computer instructions executable by the processor to determine a recommended medical device based on the received patient specific data. For example, if the patient specific data includes an X-ray of a knee for a replacement knee surgery, the computer 108 is configured to determine the implant size that would best fit into the knee. After determining the best implant size, the computer 108 outputs on, e.g., a display, the determined size for selection by the end user. If the end user selects the determined best implant size, the dispensing mechanism 104 outputs the correspondingly sized trial implants and/or kit having the correspondingly sized trial implants. Additionally, the kit having the correspondingly sized trial implants can also include additional trial implants of different sizes based on secondary sizing parameters associated with the determined implant size, such as, for example, implants marginally larger or smaller in a particular aspect, e.g., thickness.

The computer 108 can also be configured to determine a recommended attribute of the medical device. Recommended attributes of the medical device can include, for example, a size or thickness of an implant, a type of implant, or a type of instrument associated with a particular implant or medical procedure. For example, the end user may input patient specific data related to a knee orthopedic surgery into the controller 106. Computer 108 will then recommend instruments and implants associated with said knee surgery.

The computer 108 is further configured to identify the medical device that is outputted by the dispensing mechanism 104 and record the medical device data, such as the UDI. The computer 108 is further configured to associate the medical device data with the patient for which it is requested, i.e., the patient being operated on. The computer 108 may be further configured to generate a unique identification code for a particular medical device that can be associated with a specific patient and the UDI of the medical device.

The memory 124 of the computer can include an internal registry database. The internal registry database may include a plurality of user profiles that are associated with operators of the medical device dispenser. Such operators can include surgeons, nurses, and other staff within an operating room. The user profiles may include user preferences or an internal registry for tracking the usage of the medical device dispenser 100 and associating that usage with the operator. Additionally, the user profiles may include privileges for controlling what medical devices the operator may order and/or what patient specific data the operator may access through the medical device dispenser 100.

The internal registry database may further include a database for tracking the usage of the medical device dispenser 100 and for monitoring and/or tracking a stock of the medical devices currently stocked in the storage compartment 102.

The processor 122 and memory 124 are configured to execute a series of functions based on a plurality of instructions. Such instructions include: (1) receiving patient specific data; (2) receiving a unique device identification associated with each of the plurality of medical devices; (3) determining at least one of the plurality of medical devices to output based on patient specific data; (4) associating the unique device identification of the determined medical device with the patient specific data; (5) determining a recommended attribute of one of the plurality of medical devices based on the patient specific data; (6) determining, based on the patient specific data, a size, orientation and thickness of the implant, a size, orientation and thickness of the trial implant, and a type of instrument; (7) generating a unique identification code for each of the plurality of medical devices stored in the storage compartment; and (8) comparing the received user input value with the determined at least one of the plurality of medical devices to output.

The processor 122 and memory 124 can also be configured to execute additional instructions such as: (1) recording the associated unique device identification of a determined medical device with the patient specific data on the network 126; (2) recording the associated unique device identification of a determined medical device with the patient specific data on the registry on the computer 108; (3) assigning operational privileges based on an input user name and password associated with one of the plurality of user profiles; (4) associating an operation of the medical device dispenser 100 with one of the plurality of user profiles; (5) tracking when a medical device is dispensed by the dispensing mechanism; and (6) monitoring a stock of one of the plurality of medical devices and communicating with the network 126 for refilling the stock of the medical device.

It is appreciated that the computer 108 can be configured to execute the executable instructions upon command from an operator of the medical device dispenser 100 via, e.g., a prompt on the controller 106. It is also appreciated that the computer 108 may be configured to automatically execute the executable instructions when a triggering condition and/or a predetermined condition is met. For example, the computer may automatically (1) determine the at least one of the plurality of medical devices to output when patient specific data is input into the controller 106 for the first time; (2) communicate with the internal registry or network 126 for recording, logging and/or associating the unique device identification and/or unique identification code with the patient specific data when one of the plurality of medical devices is outputted by the dispensing mechanism 104; (3) adjust user settings and privileges when a user profile is accessed; (4) communicate with the network 126 for restocking the medical device dispenser 100 when one of the plurality of medical devices exceeds a minimum threshold; and so forth.

As shown in FIG. 2, the medical device dispenser 100 can further include an evaluation mechanism 128, a marking mechanism 130, a sterilization mechanism 132 and a communication module 134 in communication with computer 108.

Referring again to FIG. 1, the evaluation mechanism 128 is a mechanism for evaluating the dispensed medical device. The evaluation mechanism 128 can include, e.g., a scale 129A, a template 129B, a camera 129C, a scanner 129D, and the like. The evaluation mechanism 128 is configured to capture properties of the dispensed medical device before it is used on a patient during surgery. In other words, the medical device dispenser further comprises an evaluation mechanism configured to evaluate an outputted medical device.

As shown in FIG. 2, the marking mechanism 130 is a mechanism for marking the outputted medical device for identification during or post-surgery. Exemplary marking mechanisms 130 include printing, etching, color coding, and the like. In an exemplary embodiment, the marking mechanism 130 is configured to apply a unique identification code 131 to the dispensed medical device for identification thereafter. The marking mechanism 130 is also configured to be in communication with the computer 108 for transmitting the unique identification code 131 thereto for recording the unique identification code of the dispensed medical device in a patient's electronic medical record (“EMR”).

The sterilization mechanism 132 is a mechanism for sterilizing the outputted medical device. Such sterilization mechanisms can include, e.g., an autoclave.

The communication module 134 is a module for communication with third-party individuals outside of the surgery room. In an exemplary embodiment, the communication module 134 is configured to allow real-time communication with a specialist or an orthopedic sales representative or engineer for assisting in the operation of the medical device dispenser 100 and/or the selection of orthopedic implants. The communication module 134 can include, e.g., a speaker, a camera, and a microphone.

Referring again to FIG. 1, the medical device dispenser 100 can further include a used storage compartment 136 and be configured to receive used medical devices to be stored therein via a receiving mechanism 138.

Similar to the storage compartment 102, the used storage compartment 136 is a housing for storing the plurality of medical devices after they have been used. In other words, the used storage compartment 136 is configured to store a medical device, e.g., an implant, a trial implant, an instrument and/or a kit, after it has been used in surgery. In an exemplary embodiment, the used storage compartment 136 is separate and distinct from the storage compartment 102. Further, in an exemplary embodiment, the medical device dispenser 100 has both the used storage compartment 136 and the storage compartment 102. However, it is appreciated that the used storage compartment 136 can be located separate from the storage compartment 102

The receiving mechanism 138 is a mechanism for receiving used medical devices, e.g., implants, trial implants, instruments and kits and optionally transporting the medical device to the used storage compartment 136.

The medical device dispenser 100 can further include a receiving evaluation mechanism 140, a receiving sterilization mechanism 142 and a receiving scanner 144.

Similar to the evaluation mechanism 128, the receiving evaluation mechanism 140 is a mechanism for evaluating the returned medical device. The receiving evaluation mechanism 140 can include e.g., a scale 141A, a template 141B, a camera 141C, a scanner 141 D, and the like. The receiving evaluation mechanism 140 is configured to capture properties of the returned medical device after it is used on a patient during surgery.

Similar to the sterilization mechanism 132, the receiving sterilization mechanism 142 is a mechanism for sterilizing the returned medical device. Such sterilization mechanisms can include, e.g., an autoclave.

The receiving scanner 144 is a scanner for scanning the received medical device after it has been used on a patient. In an exemplary embodiment, the receiving scanner 144 is configured to scan the UDI and/or the unique identification code of the used medical device and communicate with the computer 108 for determining that the medical device has been returned. The receiving scanner 144 can be further configured to supplement the receiving evaluation mechanism 140 to determine if the returned medical device has been damaged.

Referring to FIGS. 1 and 2, the medical device dispenser 100 is configured as shown. The dispensing mechanism 104 is in operative communication with the storage compartment 102 and the individual storage sections holding the medical devices, e.g., the implants 110, trial implants 112, instruments 114, and kits 116. The sterilization mechanism 132, the marking mechanism 130 and the evaluation mechanism 128 are also in operative communication with the dispensing mechanism 104.

Furthermore, in an exemplary embodiment, the receiving mechanism 138 is spaced from the dispensing mechanism 104. The receiving mechanism 138 is operatively in communication with the used storage compartment 136 for storing the used medical devices, e.g., the implants 110, trial implants 112, instruments 114, and kits 116. The receiving mechanism 138 is also operatively in communication with the receiving evaluation mechanism 140, receiving sterilization mechanism 142 and receiving scanner 144.

The computer 108 is operatively in communication with the controller 106 for receiving patient specific data. The computer 108 is further in operative communication with the communication module 134, the dispensing mechanism 104, the marking mechanism 130, the evaluation mechanism 128, the sterilization mechanism 132, the input scanner 120, the network 126, the receiving scanner 144, the receiving mechanism 138, the receiving evaluation mechanism 140 and the receiving sterilization mechanism 142 for operation therewith. The computer 108 is further operatively in communication with the marking mechanism 130, the evaluation mechanism 128, the receiving scanner 144, and the receiving evaluation mechanism 140 for receiving operational metrics of the medical devices generated by the medical device dispenser 100, such as weight, condition, unique identification code, and so forth.

An exemplary embodiment of the medical device dispenser 100 operates by storing, and outputting, medical devices based on patient specific data. An operator of the medical device dispenser 100 first inputs patient specific data, such as an X-ray image, into the controller 106. The computer 108 then processes the patient specific data and determines a recommended medical device, e.g., a trial implant of a specific size, for the operator based on the received patient specific data. After confirming the recommended medical device, the dispensing mechanism 104 outputs the medical device, e.g., the trial implant 112 or kit 116 having the recommended medical device and associated instruments. When the recommended medical device is outputted, the computer 108 records the outputted medical device and associates the medical device via the UDI and/or the unique identification code with the patient specific data.

Before or during the dispensing process, the medical device dispenser 100 may sterilize the outputted medical device with the sterilization mechanism 132, may mark the outputted medical device with the marking mechanism 130, and/or may evaluate the condition, e.g., weight, of the medical device with the evaluation mechanism 128. The computer 108 then records the outgoing status of the dispensed medical device and associates the data with the patient specific data. Additionally, if necessary, the operator can use the communication module 134 to communicate with a specialist to assist in the operation of the medical device dispenser 100.

After the user receives the dispensed medical device, the user may then proceed to use it for surgery. After usage, the user may return the medical device to the medical device dispenser 100 via the receiving mechanism 138. When returned, the used medical device is re-sterilized via the receiving sterilization mechanism 132 and re-evaluated using the receiving evaluation mechanism 140.

Additionally, the receiving scanner 144 activates to scan the received medical devices. The receiving mechanism 138 can then asses all of the returned medical devices to determine if the same quantity of devices have been returned as have been outputted by the dispensing mechanism 104. After the medical device is returned, then the computer 108 can determine if any damage has been done or if a part of the device, e.g., a trial implant in a kit 116, has not been returned. After confirmation, the returned medical device(s) is stored in the used storage compartment and the data of the returned medical device is re-associated with the patient specific data.

During use, the operator uses the controller 106 to input, e.g., a desired final orthopedic size based on trialing with the trial implants. Similar to the steps described above, the desired medical device, e.g., an orthopedic implant 110, is dispensed and the medical device data, e.g., UDI, is associated with the patient specific data, along with an assigned unique identification code 131.

The advantages of having a medical device dispenser are apparent. Specifically, the medical device dispenser can be used to supplement or replace an orthopedics representative by streamlining the storage and selection of orthopedic implants. Further, the medical device dispenser can further assure that all necessary orthopedic implants are present for the surgery, as the computer can keep track of its stock.

While the subject disclosure has been described with reference to exemplary embodiments, it will be appreciated by those skilled in the art that various changes may be made and equivalents may be substituted for elements thereof without departing from the scope of the exemplary embodiments. For example, features described in one embodiment may be incorporated into a different embodiment, such as the use or non-use of a receiving mechanism. Additionally, features described in one manner may instead be accomplished by known techniques in the art, such as the type of dispensing or receiving mechanism.

In addition, modifications may be made to adapt a particular situation or material to the teachings of the various embodiments without departing from the essential scope thereof. For example, the medical device dispenser may only be configured to dispense medical devices. It is to be understood, therefore, that the exemplary embodiments not be limited to the particular aspects disclosed, but it is intended to cover modifications within the spirit and scope of the exemplary embodiments as discussed above.

1. A medical device dispenser comprising:
a storage compartment for storing a plurality of medical devices;
a dispensing mechanism for dispensing at least one of the plurality of medical devices; and
a computer that includes a processor and a memory having stored thereon computer instructions executable by the processor to:
receive patient specific data,
receive a unique device identification associated with each of the plurality of medical devices,
determine at least one of the plurality of medical devices to output based on the patient specific data, and
associate the unique device identification of the determined medical device with the patient specific data.

2. The medical device dispenser of claim 1, wherein the memory further includes computer instructions executable by the processor to determine a recommended attribute of one of the plurality of medical devices based on the patient specific data, wherein the recommended attribute is a size, a thickness, a type of implant, or a type of instrument, wherein the patient specific data includes at least one of image data, height data, and weight data, or wherein the image data is an X-ray, a CT scan or an MRI scan.

3. The medical device dispenser of claim 1, wherein the plurality of medical devices includes implants, trial implants, instruments associated with the implants, and kits having trial implants or instruments associated with the implants, and wherein the memory further includes computer instructions executable by the processor to determine based on the patient specific data:
a size, orientation and thickness of the implant,
a size, orientation and thickness of the trial implant, and
a type of instrument.

4. The medical device dispenser of claim 1, wherein the memory further includes computer instructions executable by the processor to generate a unique identification code for each of the plurality of medical devices stored in the storage compartment.

5. The medical device dispenser of claim 4, further comprising a marking mechanism for marking each of the plurality of medical devices stored in the storage compartment.

6. The medical device dispenser of claim 1, further comprising an input controller configured to receive user input values for selecting at least one of the plurality of medical devices to output from the dispensing mechanism, and wherein the memory further includes computer instructions executable by the processor to compare the received user input value with the determined at least one of the plurality of medical devices to output.

7. The medical device dispenser of claim 1, further comprising an evaluation mechanism configured to evaluate an outputted medical device, wherein the evaluation mechanism is a scale, a template or a scanner.

8. The medical device dispenser of claim 1, further comprising a communication module.

9. The medical device dispenser of claim 1, further comprising a sterilization mechanism for sterilizing one of the plurality of medical devices to be outputted.

10. The medical device dispenser of claim 1, wherein the computer is in communication with a network, wherein the network is a cloud-based network or a central server.

11. The medical device dispenser of claim 10, wherein the memory further includes computer instructions executable by the processor to record the associated unique device identification of the determined medical device with the patient specific data on the network.

12. The medical device dispenser of claim 1, wherein the memory further includes computer instructions executable by the processor to record the associated unique device identification of the determined medical device with the patient specific data on a registry database on the computer.

13. The medical device dispenser of claim 1, wherein the memory includes a plurality of user profiles, and wherein the memory further includes computer instructions executable by the processor to associate an operation of the medical device dispenser with one of the plurality of user profiles.

14. The medical device dispenser of claim 1, wherein the memory further includes computer instructions executable by the processor to assign operational privileges.

15. The medical device dispenser of claim 1, wherein the memory further includes computer instructions executable by the processor to track a medical device dispensed by the dispensing mechanism.

16. The medical device dispenser of claim 1, wherein the memory further includes computer instructions executable by the processor to monitor a stock of one of the plurality medical devices.

17. The medical device dispenser of claim 1, wherein the computer is configured to automatically execute predetermined instructions when a triggering condition is met.

18. An orthopedic medical device dispenser comprising:
a storage compartment for storing a plurality of medical devices including:
a plurality of orthopedic implants,
a plurality of orthopedic trials, and
a plurality of orthopedic instruments associated with the plurality of orthopedic implants;
a dispensing mechanism for dispensing at least one of the plurality of medical devices; and
a computer that includes a processor and a memory having stored thereon computer instructions executable by the processor to:
receive patient specific data,
receive a unique device identification associated with each of the plurality of orthopedic implants,
determine at least one of the plurality of medical devices to output based on the patient specific data, and
associate the unique device identification of the determined orthopedic implant with the patient specific data.

19. The orthopedic medical device dispenser of claim 18, wherein the memory further includes computer instructions executable by the processor to:
determine at least one of the plurality of orthopedic implants to output based on the patient specific data,
determine at least one of the plurality of orthopedic trials to output based on the patient specific data, and
determine at least one of the plurality of orthopedic instruments associated with the plurality of orthopedic implants to output based on the patient specific data.

20. A method of dispensing an orthopedic implant comprising the steps of:
inputting patient specific data into a medical device dispenser, the medical device dispenser including:
a storage compartment for storing a plurality of orthopedic implants,
a dispensing mechanism for dispensing at least one of the plurality of orthopedic implants,
an input controller configured to receive user input values for selecting at least one of the plurality of orthopedic implants to output from the dispensing mechanism, and
a computer that includes a processor and a memory having stored thereon computer instructions executable by the processor to:
receive the patient specific data,
receive a unique device identification associated with each of the plurality of orthopedic implants,
determine at least one of the plurality of orthopedic implants to output based on the patient specific data,
associate the unique device identification of the determined orthopedic implant with the patient specific data, and
output on a display the determined orthopedic implant to be outputted,
confirming, using the input controller, the determined orthopedic implant to be outputted;
receiving the outputted orthopedic implant outputted by the dispensing mechanism; and
using the computer, generating a unique identification code and associating the unique identification code and unique device identification with the patient specific data.
